(19) United States
(12) Patent Application Publication
(10) Pub. No.: US 2007/0010697 A1
(43) Pub. Date: Jan. 11, 2007

(54) Method for producing tertiary butyl alcohol

(75) Inventors: Atsushi Koizumi, Otake-shi (JP); Akira Ogawa, Tokyo (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Mitsubishi Rayon Co., Ltd., Tokyo (JP)

(21) Appl. No.: 10/568,614

(22) PCT Filed: Aug. 18, 2004
(86) PCT No.: PCT/JP04/12139

§ 371 Date: Aug. 23, 2006

(30) Foreign Application Priority Data
Aug. 21, 2003 (JP) 2003-297323

(51) Int. Cl. C07C 29/03

(52) U.S. Cl. 568/697

(57) ABSTRACT

The present invention provides a method for producing a tertiary butyl alcohol with a high reaction rate of hydration of isobutylene. Furthermore, the present invention provides a method for producing a tertiary butyl alcohol with a high reaction rate of hydration of isobutylene even in the case of using an isobutylene of low concentration as a raw material. In this method, a tertiary butyl alcohol is produced from isobutylene and water in the presence of a cation-exchange resin catalyst and at least one solvent selected from the group consisting of sulfones and organic carboxylic acids by using a catalytic distillation apparatus. The solvent to be used in the method for producing a tertiary butyl alcohol is preferably sulfolane, dimethyl sulfone or acetic acid.

### TECHNICAL FIELD

The present invention relates to a method for producing tertiary butyl alcohol.

The present application claims the priority of Japanese Patent Application No. 2003-297323 filed on Aug. 21, 2003, the contents of which are incorporated herein by reference.

### BACKGROUND ART

Until now, a method for producing tertiary butyl alcohol through a conventional hydration reaction of isobutylene and water in the presence of a cation-exchange resin catalyst has been known. For example, in Patent document 1, a method for producing tertiary butyl alcohol by using a catalytic distillation apparatus is disclosed. And in Patent document 2, a method for producing tertiary butyl alcohol by using sulfolane as a solvent for the hydration reaction is disclosed.

Patent document 1: Japanese Patent Application, First Publication No. Hei 3-106840

Patent document 2: Japanese Patent Application, First Publication No. Hei 8-53381

In the method disclosed in Patent document 1, there was a problem that a large amount of the catalyst was necessary for the method to be industrially viable, because a contact among a water phase, an oil phase composed of a hydrocarbon containing isobutylene and a cation-exchange resin was insufficient so that the rate of a hydration reaction became low. In addition, in the method disclosed in Patent document 2, there was a problem that a productivity was low (namely, a yield was low) when an isobutylene of low concentration was used as a raw material, because a hydration reaction is an equilibrium reaction so that a conversion of isobutylene is restricted.

This invention has been achieved taking the above-mentioned problems into consideration and an object is to provide a method for producing tertiary butyl alcohol wherein the rate of a hydration reaction of isobutylene is high, especially when an isobutylene of low concentration is used as a raw material.

### DISCLOSURE OF INVENTION

To solve the problems described above, the present inventors have intensively researched a hydration reaction of isobutylene and have found that by using a catalytic distillation apparatus and a specific solvent, the reaction rate is dramatically improved and the amount of catalyst can be sharply reduced as compared with a conventional method even in the case of using an isobutylene of low concentration as a raw material, and thus have completed the present invention.

In other words, the present invention is a method for producing a tertiary butyl alcohol from isobutylene and water in the presence of a cation-exchange resin catalyst and at least one solvent selected from the group consisting of sulfones and organic carboxylic acids by using a catalytic distillation apparatus.

As for the solvent, at least one solvent selected from the group consisting of sulfolane, dimethyl sulfone and acetic acid is preferable and the amount of the solvent is preferably 0.01 mol or more to 1 mol of isobutylene.

As for the cation-exchange resin, polystyrenesulfonic acid resin, phenolsulfonic acid resin or perfluorosulfonic acid resin as well as a porous cation-exchange resin is preferable.

### BRIEF DESCRIPTION OF DRAWINGS

FIG. 1 is a schematic diagram showing a catalytic distillation apparatus.

### BEST MODE FOR CARRYING OUT THE INVENTION

As a raw isobutylene, isobutylene alone may be used, however, a liquefied gas composed of a hydrocarbon containing isobutylene (hereinafter referred to as a liquefied gas) is generally used. As a hydrocarbon containing isobutylene, a mixture such as butenes containing isobutylene, butanes or the like is exemplified. A mixture of C4 hydrocarbons obtained through thermal cracking, steam cracking or catalytic cracking of petroleum compounds, preferably a butadiene-removed residue thereof is commercially used. The isobutylene concentration in the mixture of C4 hydrocarbons is not particularly limited, but is generally 80% by mass or less with respect to the mixture commercially available. The isobutylene concentration in the liquefied gas is preferably 5 to 60% by mass and more preferably 10 to 60% by mass in view of the availability of the raw materials and the improvement of the reaction rate.

Further, a liquefied gas containing an unreacted isobutylene remaining after the hydration reaction can be used as a raw isobutylene. In this case, the isobutylene concentration in the mixture of C4 hydrocarbons is generally reduced to a considerably low value such as 5 to 15% by mass owing to the consumption of the isobutylene, however, the raw isobutylene can be satisfactorily supplied to the reaction according to the method of the present invention. The liquefied gas containing the unreacted isobutylene usually forms a mixture with an aqueous solution of tertiary butyl alcohol, but the mixture can be used as the raw isobutylene without subjecting to a separation. The raw water is not particularly limited and distilled water, for example, demineralized water, drinking water or the like can be used. The amount of water is preferably 1.0 to 10 mole and more preferably 1.05 to 8 mole to 1 mole of isobutylene from the viewpoint of suppression of formation of a dimer and trimer of isobutylene and an improvement of the reaction rate.

The solvent to be used in the present invention is at least one solvent selected from the group consisting of sulfones and organic carboxylic acids. The solvent may be a mixed solvent consisting of two or more of the solvents in the group or a mixed solvent containing a solvent not included in the group.

As a solvent for the sulfones, for example, sulfolane, 2-methyl sulfolane, 3-methyl sulfolane, 3-propyl sulfolane, 3-butyl sulfolane, dimethyl sulfone, diethyl sulfone, methyl ethyl sulfone, dipropyl sulfone, sulfonal, trional and the like are exemplified.

As a solvent for the organic carboxylic acids, including carboxylic acid anhydrides, for example, acetic acid, acetic anhydride, propionic acid, propionic anhydride, butyric acid, isobutyric acid and the like are exemplified.

Among them, sulfolane, dimethyl sulfone and acetic acid are preferable as a solvent to be used in the present invention.

The amount of solvent is preferably 0.01 mole or more and still preferably 0.05 mole or more to 1 mole of isobutylene to be supplied to the catalytic distillation apparatus as the lower limit from the viewpoint of the reaction rate and profitability. On the other hand, the amount of solvent is preferably 1.0 mole or less and still preferably 0.8 mole or less as the upper limit.

As a cation-exchange resin to be used in the present invention, a strongly acidic cation-exchange resin is preferable from the viewpoint of improvement of the reaction rate. As a strongly acidic cation-exchange resin, a polystyrenesulfonic acid resin which is composed of a copolymer of styrene and divinyl-benzene added with a sulfonic acid group, a phenolsulfonic acid resin which is composed of a condensated material of phenol and formaldehyde added with a sulfonic acid group, and a perfluorosulfonic acid resin which is composed of a copolymer of fluorovinyl ether and fluoro carbon added with a sulfonic acid group are exemplified.

Further, a cation-exchange resin is classified into a gel type, a porous type which has physical micro-pores and the like according to their geometrical structure, and a porous cation-exchange resin is preferable in view of the improvement of the reaction rate. As the porous cation-exchange resin, for example, a trade name “Lewatit” manufactured by Bayer AG, trade name “Amberlite” manufactured by Rohm & Haas Corporation and the like are exemplified.

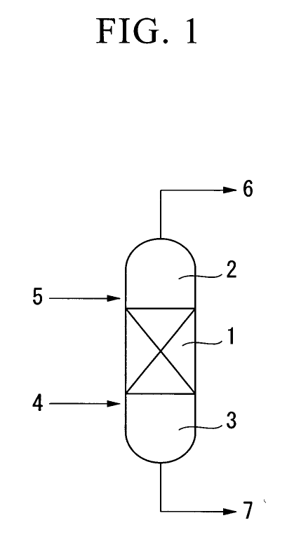

The catalytic distillation apparatus to be used in the present invention is provided with a reaction part in which a catalyst exists and a reaction and a distillation can be performed simultaneously. As such a catalytic distillation apparatus, for example, a type of apparatus is exemplified in which a reaction part 1 packed with a cation-exchange resin is provided as shown in FIG. 1, the reaction part being able to perform a reaction and a distillation simultaneously. The catalytic distillation apparatus is preferably provided with a concentrating part 2 in the upper stage of the reaction part, a recovering part 3 in the lower stage of the reaction part, a reboiler and a condenser (in FIG. 1, the reboiler and the condenser are omitted). Further, a decanter for separating water may be provided downstream of the condenser as occasion demands.

A packing method of the cation-exchange resin is not particularly limited. However, because the cation-exchange resin is usually a particle having a diameter of around 0.3 to 1.2 mm, the pressure drop becomes large when the particles are packed in a tower. Consequently, it is preferable to increase a void fraction by using, for example, the following methods: a method to keep the cation-exchange resin in a basket-like pouch, the shape of which is a cylindrical type, spherical type, doughnut-like type, cubic type or tubular type woven with stainless steel, nylon, glass fiber, polyester, cotton or the like; a method to keep the cation-exchange resin sandwiched between corrugated sheets woven into wire mesh; a method to keep the cation-exchange resin in a wire mesh container set on a tray; and a method to pack the cation-exchange resin together with another packing material such as a Raschig ring, Berl saddle or the like. Further, the void fraction is preferably 60 to 95%.

For example, in the catalytic distillation apparatus of FIG. 1, it is preferable that, in the reaction part 1, the raw isobutylene is supplied from a part 4 located lower than the reaction part and the raw water is supplied from a part 5 located higher than the reaction part so that the raw materials and the cation-exchange resin can come in contact effectively. On the other hand, a method of supplying the solvent is not particularly limited, however, in case that the boiling point of the solvent is higher than that of water, the solvent is preferably supplied from the part located higher than the reaction part and in case that the boiling point of the solvent is lower than that of water, the solvent is preferably supplied from the appropriate part located in accordance with the boiling point.

The temperature of the reaction part (the reaction temperature) when the hydration reaction is performed is preferably 25 to 100° C., more preferably 45 to 95° C., because a side reaction is liable to occur in case that the reaction temperature becomes higher than it needs, and the reaction rate is lowered in case that the reaction temperature becomes lower than it needs. The reaction pressure is equivalent to the vapor pressure corresponding to the reaction temperature of the liquefied gas which consists of hydrocarbons including isobutylene, and is preferably 0.2 to 2 Mpa, more preferably 0.4 to 1.6 Mpa.

When performing the reaction, it is preferable that a portion of the liquid obtained by condensing a vapor 6 of the column top with a condenser is drawn out as a distillate and the remainder is recycled to the column.

Further, a bottom liquid 7 containing tertiary butyl alcohol is preferably drawn out continuously. The drawn out liquid can be separated into tertiary butyl alcohol and the solvent through an alcohol separating column and the like as occasion demands. The tertiary butyl alcohol thus obtained becomes a product. Furthermore, the separated solvent may be returned to the catalytic distillation apparatus and repeatedly used.

As a method for making the solvent exist in the catalytic distillation apparatus, for example, a method of supplying the solvent alone to the column, a method of supplying the solvent and the raw isobutylene or raw water in the form of a mixed liquid into the column and other methods are exemplified.

### EXAMPLES

Hereinafter, the present invention will be entered into details with reference to the following examples, however, these examples represent merely concrete examples of the present invention and the present invention is not limited to these examples.

(Analytical Method of Isobutylene)

Gas chromatograph: trade name “HP6850 Series” manufactured by Hewlett Packard

Column: trade name “AL2O3/KCL PLOT” manufactured by GL Science; internal diameter 0.32 mm; length 50 m; DF 5.0, 100° C.×5 min, 20° C./min, 150° C.×5 min

Detector: TCD, 200° C.

Inlet: 200° C.; sprit ratio 1/20; total flow 22 mL/min; inlet pressure 82 kPa; injection amount 100 μL

The calibration curve was prepared in the following manner: sampling an optional mixture of isobutylene and isobutane in a pressure vessel, then attaining a vapor-liquid equilibrium of the system under a constant temperature, then sampling a small amount of the vapor phase into a gasbag, then analyzing isobutylene and isobutane in the gas with gas chromatography, then changing the composition of the mixture of isobutylene and isobutane and following the same procedure as the above, and obtaining the relationship between the isobutylene/isobutane mass ratio and the isobutylene/isobutene area ratio.

(Analytical Method of Tertiary Butyl Alcohol and Isobutylene Dimer)

The analysis was performed by gas chromatography.

Gas chromatograph: trade name “GC-14B” manufactured by Shimadzu Corporation

Column: trade name “DB-WAX” manufactured by J & W Corporation, internal diameter 0.25 mm; length 30 m; DF 0.25 μm, 50° C.×5 min, 20° C./min, 150° C.×5 min

Detector: FID, 200° C., split-less

Inlet pressure 100 kPa; injection amount 1 μL

### Example 1

The catalytic distillation apparatus is configured with a reboiler part, a reaction part and a condenser part in this order from the bottom, and these are connected with conduits. A 1 L glass autoclave equipped with a stirrer, a thermometer, a pressure gauge and a heater for heating (trade name “HAIPA-GURASUTA-TEM-V type” manufactured by TAIATSU TECHNO CORPORATION) was used as the reboiler part. And a stainless steel tubular reactor having an internal diameter of 28 mm and a length of 240 mm equipped with temperature sensors at a column top and a column bottom was used as the reaction part. And a stainless steel double pipe condenser having an internal diameter of 28 mm and a length of 200 mm was used as the condenser part.

As a catalyst of a cation-exchange resin, a strongly acidic macro-porous ion-exchange resin “Lewatit Catalyst K2621” manufactured by Bayer AG was used. The catalyst was contained in a small bag made of wire mesh of 80 mesh (8 mm in internal diameter, 40 mm in length) and packed into the reaction part and used.

As a solvent, trade name “sulfolane” manufactured by Wako Pure Chemical Industries, Ltd. was used. The following method of adding the solvent was performed. About 200 g of 40% by mass of sulfolane aqueous solution was prepared in a 500 cm³ container, into which an ion-exchange resin dried for 24 hours in a hot air dryer of 50° C. and contained in a small bag was soaked for 30 minutes, and the ion-exchange resin was swollen with the sulfolane aqueous solution. Then, the small bag was pulled up and an excess of the sulfolane aqueous solution was removed by blowing pressurized air. From the change of the weight between before and after the swelling, it was calculated that 10.3 g (0.086 mol) of sulfolane and 15.5 g (0.860 mol) of water was contained in the ion-exchange resin (48 cm³ of the dried material). The above ion-exchange resin (wet) was packed in a tubular reactor. Then, the void fraction of the reaction part based on the dried material was 63% calculated from a packing volume of 130 cm³ of the reaction part.

Into the glass autoclave, 20.5 g (0.365 mol) of liquefied isobutylene and 170 g of liquefied butane were introduced (then, the isobutylene concentration in the C4 mixture was 2.1% by mass), and subjected to stirring and heating to start the reaction. The gas vaporized at the reboiler part was supplied to the reaction part located at the upper part and then introduced to the condenser part where the condensed liquefied gas was all refluxed to the reaction part, and further returned to the reboiler part. Because the ion-exchange resin was packed in the reaction part, the reaction was performed in the state of a gas-liquid mixed phase. Further, the tertiary butyl alcohol produced in the reaction part was quickly moved to the reboiler part owing to the effect of the distillation.

The temperature of the cooling water of the condenser was controlled so as to make the reaction pressure and the bottom temperature 1.0 Mpa and 70° C., respectively, during the reaction. When these conditions were stable, the temperature of the cooling water of the condenser was in the range from 33 to 35° C. Further, the amount of vapor in the system was adjusted to become 40 cm³/min by controlling the voltage of the heater.

The curves representing the variation with time of the molar fraction of isobutylene in the C4 hydrocarbon mixture in the reboiler and the amount of isobutylene were made by sampling the gas at a fixed interval from the start of the reaction and obtaining the amount of isobutylene with gas chromatography. Using these as a basis, the consumption rate of isobutylene per unit amount of the catalyst to the molar fraction of isobutylene was calculated. The result is shown in Table 1.

The reaction was finished after 5.5 hours had passed from the start of the reaction and heating was stopped. Then, the system was cooled and the C4 hydrocarbon mixture was blown out and the reaction product remaining in the autoclave was analyzed to show that 0.16% by mass of isobutylene dimer was detected and isobutylene other than the dimer was changed into tertiary butyl alcohol.

TABLE 1

| | Example 1 | | Example 2 | | Example 3 | | Comparative |
|---|---|---|---|---|---|---|---|
| | sulfolane | | dimethyl sulfone | | acetic acid | | example |
| | Consumption | | Consumption | | Consumption | | Consumption |
| Molar | rate of | | rate of | | rate of | | rate of |
| fraction of | isobutylene | Compared to | isobutylene | Compared to | isobutylene | Compared to | isobutylene |
| isobutylene | [mol/(L- | comparative | [mol/(L- | comparative | [mol/(L- | comparative | [mol/(L- |
| [−] | catalyst · hr)] | example | catalyst · hr)] | example | catalyst · hr)] | example | catalyst · hr)] |
| 0.11 | 3.0 | (1.42) | 2.8 | (1.30) | 2.9 | (1.37) | 2.1 |
| 0.10 | 2.7 | (1.39) | 2.5 | (1.26) | 2.7 | (1.36) | 1.9 |
| 0.09 | 2.4 | (1.35) | 2.2 | (1.22) | 2.4 | (1.36) | 1.8 |
| 0.08 | 2.1 | (1.30) | 1.9 | (1.16) | 2.2 | (1.36) | 1.6 |
| 0.07 | 1.8 | (1.25) | 1.6 | (1.09) | 1.9 | (1.36) | 1.4 |

### Comparative Example

The procedure was carried out in the same manner as in Example 1 except that sulfolane was not added. The result is shown in Table 1. The consumption rate of isobutylene per unit amount of the catalyst was low because the solvent was not added.

### Example 2

The procedure was carried out in the same manner as in Example 1 except that 5.2 g of trade name “dimethyl sulfone” manufactured by Wako Pure Chemical Industries, Ltd. and 17.8 g of water were used. The result is shown in Table 1.

### Example 3

The procedure was carried out in the same manner as in Example 1 except that 6.4 g of trade name “acetic acid” manufactured by Wako Pure Chemical Industries, Ltd. and 13.3 g of water were used. The result is shown in Table 1.

### INDUSTRIAL APPLICABILITY

Using the method of the present invention, tertiary butyl alcohol can be produced with a high reaction rate of hydration of isobutylene. Furthermore, even in the case of using an isobutylene of low concentration as a raw material, tertiary butyl alcohol can be produced with a high reaction rate of hydration of isobutylene.

What is claimed is:

1. A method for producing a tertiary butyl alcohol from isobutylene and water in the presence of a cation-exchange resin catalyst and at least one solvent selected from the group consisting of sulfones and organic carboxylic acids by using a catalytic distillation apparatus.

2. The method for producing a tertiary butyl alcohol according to claim 1, wherein the solvent is sulfolane, dimethyl sulfone or acetic acid.

3. The method for producing a tertiary butyl alcohol according to claim 1, wherein the amount of solvent is 0.01 mole or more to 1 mole of isobutylene.

4. The method for producing a tertiary butyl alcohol according to claim 1, wherein the cation-exchange resin is polystyrenesulfonic acid resin, phenolsulfonic acid resin or perfluorosulfonic acid resin.

5. The method for producing a tertiary butyl alcohol according to claim 1, wherein the cation-exchange resin is a porous cation-exchange resin.
